(19) United States
(12) Patent Application Publication
(10) Pub. No.: US 2022/0226248 A1
(43) Pub. Date: Jul. 21, 2022

(54) Pharmaceutical Dosage Forms and Method For Their Production

(71) Applicant: DiHeSys Digital Health Systems GmbH, Schwäbisch-Gmünd (DE)

(72) Inventors: Markus Dachtler, Ulm (DE); Gerald Huber, Plüderhausen (DE)

(21) Appl. No.: 17/615,450

(22) PCT Filed: May 29, 2020
(86) PCT No.: PCT/EP2020/065097

§ 371 Date: Nov. 30, 2021

(30) Foreign Application Priority Data
May 31, 2019 (EP) 19177751.5

(51) Int. Cl. A61K 9/20

(52) U.S. Cl. CPC A61K 9/2072; A61K 9/2095

(57) ABSTRACT

The invention relates to a method for producing solid or semi-solid dosage forms of pharmaceutical active ingredients. According to the method, an active-ingredient-free carrier structure is arranged in a 2D or 3D printing device and at least one pharmaceutical active ingredient is applied to at least one portion of the carrier structure by way of a 2D or 3D printing method carried out by the printing device. The invention also relates to semi-solid or solid dosage forms that are producible by the method according to the invention.

### FIELD OF THE INVENTION

The present invention relates to methods for producing solid or semisolid dosage forms of pharmaceutical active agents in which an active agent-free carrier structure is provided in a 2D or 3D, respectively, printing device and at least one pharmaceutical active agent is applied onto at least a region of the carrier structure through a 2D or 3D, respectively, printing process by means of said printing device. The invention also relates to semisolid or solid dosage forms producible by the method of the invention.

### BACKGROUND OF THE INVENTION

An additive 3D printing process for producing pharmaceutical dosage forms by means of fused filament fabrication (FFF) is known from WO 2016/038356 A1. In a similar process other forms such as granules, pellets, powders or flakes are used instead of filaments as starting materials which in turn are printed in the form of filaments (fused layer modeling, FLM).

The technical problem of the present invention is the provision of methods for producing pharmaceutical dosage forms which methods enable the combination of the advantages of classical production of solid or semisolid, respectively, dosage forms with those of the production of dosage forms by additive processes.

The above technical problem is solved by the embodiments of the present invention as disclosed in the claims, the present description and the accompanying drawings.

### SUMMARY OF THE INVENTION

In particular, the present invention provides a method for producing a solid or semisolid pharmaceutical dosage form containing an active agent-free carrier structure and at least one pharmaceutical active agent which is provided on at least a region of the carrier structure, the method comprising:

(i) providing at least one active agent-free carrier structure in a printing device designed for 2D and/or 3D printing of at least one pharmaceutical agent; and

(ii) applying at least one pharmaceutical active agent by 2D and/or 3D printing on at least one region of the surface of the carrier structure in the printing device.

According to the invention the application of one or more pharmaceutical agents on an agent-free carrier is carried out in the present method by means of an additive production process which is provided as a two-dimensional (2D) or three-dimensional (3D) printing process wherein 2D and 3D printing processes can also be combined.

In an embodiment of the method the agent is present in a filament in a meltable filament carrier substance or filament base substance whereby it can or is, respectively also intended that more than one agent are used, which agents can be present together in one filament or in different filaments. Such meltable active agent-containing filaments are printed in this embodiment according to a per se known FFF (fused filament fabrication) process as described, e.g. in WO 2016/038356 A1, at least in regions onto the active agent-free carrier structure. In another embodiment the meltable carrier substance or base substance, respectively, containing the agent or agents, is present in a different form, preferably as granules, pellets, powders or flakes, which are then printed by the per se known FLM (fused layer modelling), typically by hot melt extrusion using a printing device designed for the FLM process, in the form of filaments at least onto regions of the carrier structure.

In another embodiment a per se known binder printing method as described, e.g. in EP 2 968 994 A1, can be used for printing, preferably 3D printing, the pharmaceutical active agent or agents. In this embodiment the active agent(s) are present in a powder containing—besides the active agent(s)—pharmaceutically acceptable additives such as binders, antioxidants, perfumes, sweeteners, or similar substances adapted to binder jetting methods. A layer of the powder is applied onto the carrier structure whereafter a binder fluid is applied to the powder layer by the printing device wherein it is also possible to apply the binder fluid suitably in pointwise or region wise fashion in those locations where the powder is designated to form the arrangement of the active agent on the active agent-free carrier substance by means of the binder fluid. Optionally, excess powder is then removed. The steps of applying the powder layer, at least pointwise or region wise application of the binder fluid and, optionally, removing excess powder are repeated, optionally using powders containing one or more other pharmaceutical active agents, until the predetermined three-dimensional arrangement of the active agent or agents, respectively, is obtained on the active agent-free carrier structure. In a final step, optionally present unbound powder can be removed.

According to a further preferred embodiment of the present invention the additive method for applying the active agent or agents is carried out in such a way that the three-dimensional formation of the active agent-containing structure on the carrier is done by preferably layer wise application of single volume increments (hereinafter also denoted as “voxels”) of a fluid wherein at least part of the applied volume increments of the fluid contain the active agent(s), and the volume increments are preferably applied layer wise in such a manner that they form an at least semisolid, preferably solid, active agent-containing structure at least region wise on the active agent-free carrier structure. The fluid can be e.g., a molten material or at least flowable material or a liquid, wherein the one or more active agent-containing volume increments contain the active agent(s) in the molten or at least flowable material, or dissolved, dispersed, or emulsified in the liquid.

The applied volume increments can be defined in an essentially free fashion and can take the form of e.g., droplets, spheres, points, cylinders, cubes, cuboids or other forms. Preferred voxel forms are e.g., droplets and spherical voxels. The shape (such as the above examples) and size of the volume of the volume increments can be combined essentially freely and independently from one another.

The binding between the single applied volume increments can be accomplished in different ways. In one embodiment the binding between such voxels may occur, for example when using a meltable material, through solidification after application on the carrier structure wherein the solidification can be carried out through different mechanisms such as simple cooling and/or chemically by known substances. In a different embodiment a suitable binder can be added to the voxel material e.g., a dispersion or a solution, which binder hardens the voxel after application, wherein the hardening by binder can occur e.g., through heat which can be applied by a suitable heat source in the printing device such as a light source, preferably a laser device. The hardening by the binder can also occur chemically by corresponding starter molecules and/or light of a suitable wavelength wherein the latter is preferably emitted again by means of a laser device. In a further embodiment the fluid of the volume increment can contain one or more starting compounds, typically monomers of one or more polymers, and after application of the voxel a polymerization is started by suitable means such as light, heat or other polymerization starters, which hardens the voxel and binds it to neighboring voxels.

In a further preferred embodiment, a two-dimensional arrangement of a substance or composition containing the agent(s) is applied, i.e. printed, onto the carrier structure. The 2D printing process can, as disclosed above, also be combined with the described 3D printing processes e.g., by firstly applying, preferably through one of the processes described above, at least region wise a three-dimensional active agent-containing arrangement onto which a final two-dimensional arrangement containing or not containing active agent(s) is then applied. According to a preferred embodiment, in particular int a 2D printing process, a fluid containing the active agent(s), preferably an active agent(s)-containing solution, emulsion or suspension, is applied, i.e. printed, region wise. After application of the fluid such as an active agent-containing solution, emulsion or suspension, part of the fluid, e.g., the solution or suspension or emulsion, respectively, will typically be absorbed by the carrier structure and another part remains on the surface of the carrier. It follows an evaporation of the (remaining) fluid (such as a solution, suspension, or emulsion) whereafter the solid, at least semisolid, component remains on the carrier structure, which component is now loaded with active agent(s).

Suitable carrier or base, respectively, materials, particularly for the preferred voxel printing method but also for the FLM/FFF processes, in which the pharmaceutical active agent(s) are present are e.g., carriers suitable for hot melt extrusion (HME) such as low melting waxes and polymers. Apart from the low melting carrier, the HME mixture can contain further process agents and aids such as binders, emollients, antioxidants, perfumes, sweeteners or similar. Suitable HME carriers and emollients are disclosed e.g., in Crowley et al. (2007) Drug Development and Industrial Pharmacy, 33, pages 909-926 (carriers: pages 917 to 919, in particular Table 1; emollients: pages 917 and 920, in particular Table 2), whereby it is espressis verbis referred to the above-mentioned sections in the present description.

The mentioned additive processes, in particular the voxel printing process (or “voxel jetting”), are generally and preferably carried out by computer assistance. Thus, a calculated two- or three-dimensional picture of the object to be printed, at least, however, of the material arrangement containing the active agent(s), is created e.g., by means of a typical CAD program. The computer-generated representation of the dosage form or at least of the active agent-containing arrangement can also be attained by scanning of a pre-existing dosage form. In the case of the preferred voxel printing, the computer-generated model picture is then subdivided into the desired, in principle freely selectable volume increments (voxels) wherein the resolution of the real dosage form will be higher the smaller the volume increments are. An active agent an/or a color substance as well as further optionally required materials and their amounts (concentration in the volume increment) can be assigned to each individual volume increment which is ultimately printed. Suitable printing devices for voxel printing are described in e.g., US 2017/03,68755 A1 and U.S. Pat. No. 6,070,107. In the case of FFF or FLM processes the arrangement or dosage form, respectively, to be printed is subdivided by computer assistance into corresponding filaments.

In a preferred embodiment of the invention, more than one active agent, i.e. two or more active agents, are applied in step (ii), wherein the active agents can be printed together in a single step or in several separate steps. In this embodiment the active agents can be applied onto the same or different regions of the carrier structure. In this embodiment the active agents can be applied within a region separated from one another or spatially one above of the other.

In a particularly preferred embodiment, the method according to the invention further comprises the application of at least one colored substance by use of 2D and/or 3D printing onto at least one region of the carrier structure in a manner such that the applied substance forms at least one information structure visible on the carrier structure. Thereby, the colored substance(s) can be applied separately form the active agent(s). It is preferred to apply the color substance(s) together with the pharmaceutical active agent(s). In an embodiment a respective substance can thus label the region or regions, respectively, where the respective active agent has been applied onto. This embodiment can therefore provide information on the active agents printed on the active agent-free carrier and their distribution in the complete dosage form. In a further development of this embodiment of the invention, the active agent-containing regions can contain different amounts or concentration(s) of the active agent which is reflected by the concentration of the respective color substance. It is to be understood that different color substances can be mixed (for example, in a filament in the case of the FLM/FFF process, a powder in the case of binder jetting, or in a voxel of the preferred embodiment) such that, by respective selection of the mixture(s), the complete visible spectrum can be used.

According to the invention, the term “color substance” comprises also luminescent, particularly fluorescent, substances.

The information structure formed by or generated by, respectively, the color substance(s) can display various information whereby several different information structures can be used by different color substances and/or by different amounts of the same color substance which can be essentially freely chosen and combined. It is particularly intended according to the invention that the at least one information structure encodes information on the kind or the nature of the active agents applied onto the carrier structure and/or on the amount(s) of the active agent(s) applied onto the carrier structure and/or on the intended time point or intended time period of taking of the dosage form and/or the intended date of taking of the dosage form and/or on patient-related data (such as name, age, sex, medication, and diseases of the patient) and/or on the cost center and/or on the attending physician and/or on the pharmaceutical company providing the dosage form and/or on the medical unit dispensing the dosage form.

The information structure can be selected from a broad range of applications. Thus, the substance(s) can be printed in the form of QR codes, characters and/or numbers. It is understood that also different patterns such as lines, grids, points, two-dimensional patterns etc. can be printed, whereby the preferred embodiment of voxel printing provides generally the most diverse possibilities. The printed image of the code can therefore contain the active agent and at the same time encode, as described above, the desired data on patient, physician, pharmacist and/or qualified medical or pharmaceutical professionals.

The skilled person understands that depending on the selected specific additive production process, the optionally present color substance(s) can be present in the respective base composition together with the pharmaceutical active agent(s). Thus, in the case of the FLM/FFF process the color substance(s) are e.g., present together with the active agent(s) in the filament carrier or base substance of the printed filament(s). The same applies for the powder in the case of the binder jetting process. Also, in the case of voxel printing which is preferred according to the invention, the color substance (or more thereof) is (are) present together with the active agent(s) in the molten base substance or in the solution or dispersion which is intended for a selected volume increment.

By using the method of the invention, various solid or semisolid dosage forms such as tablets e.g., oblong shape tablets, lozenges, implantable tablets, multiapplication tablets, disperse tablets, retard tablets, vaginal tablets, eye tablets, coated tablets, matrix tablets, chewable tablets, film-coated tablets, modified-release tablets, lacquer tablets, and enteric coated tablets, capsules, plasters, suppositories, or thin films such as ODF (orally dissolvable film or orally degradable film) products can be produced.

According to the invention also the carrier structure can be produced through an additive production process whereby the methods of FLM/FFF printing, binder jetting and/or voxel printing as outlined above can be used. According to the invention, the carrier structure can therefore also be produced firstly by such a process in a corresponding printing device and provided in such a manner that the active agent-containing arrangement can be applied thereafter. Alternatively, the carrier structure can be generated by a conventional production process for pharmaceutical dosage forms, then provided in a suitable 2D or 3D printing device (particularly preferred in a printing device designed for voxel printing), and ultimately the active agent-containing arrangement is generated on the carrier structure.

A further aspect of the invention is semisolid or solid dosage forms, preferably the dosage forms mentioned above, producible by the method according to the invention.

Accordingly, the invention relates to a solid or semisolid dosage form comprising an active agent-free carrier structure onto which at least one pharmaceutical active agent is applied at least in regions thereof in a two- and/or three-dimensional arrangement. As outlined above, also more than one pharmaceutical active agent (i.e., two or more active agents) can be applied on the same or different regions of the carrier structure. As already outlined above for the method, the active agents can be applied within a region separated from one another or spatially one above the other.

Preferably, the dosage form also has a colored substance applied at least on a region of the carrier substance such that the applied substance can form at least one visible information structure on the carrier structure. Preferred embodiments of this aspect have already been outlined in the context of the method according to the invention. In particular, the at least one information structure can encode information contained therein relating to the kind or the nature of the active agents applied onto the carrier structure and/or the amount(s) of the active agent(s) applied onto the carrier structure and/or on the intended time point or intended time period of taking of the dosage form and/or the intended date of taking of the dosage form and/or on patient-related data and/or on the cost center and/or on the attending physician and/or on the pharmaceutical company providing the dosage form and/or on the medical unit dispensing the dosage form. Patient-related data are preferably selected from name, age, sex, medication, and diseases of the patient.

As already outlined above, the dosage form can contain various information structures, preferably those described above for the method.

### BRIEF DESCRIPTION OF THE DRAWINGS

FIGS. 1A to 1D show schematic representations of top views of different exemplary dosage forms of the invention, wherein active agent-containing arrangements comprising patient-related data or information, respectively, on the time point of taking, or similar were printed onto a flat active agent-free carrier structure. 1 Name or address of patient; 2 enlargement of printing space for higher resolution for dynamic dosing; 3 batch number or patient ID; 4 personalized note for time point of taking.

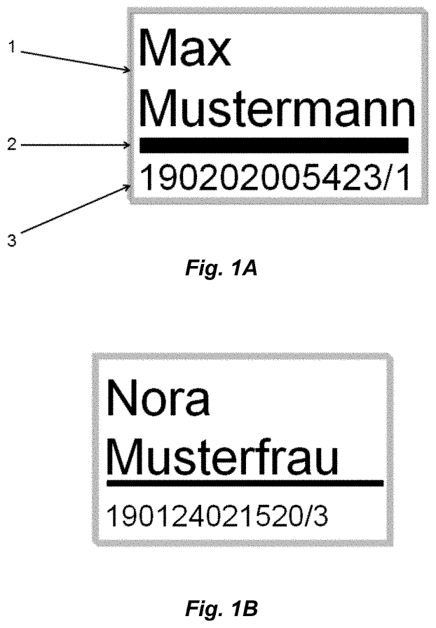

FIG. 2 shows a photographic representation of a top view of an exemplary dosage form of the invention, wherein an active agent-containing arrangement comprising a QR code was printed on an active agent-free carrier. An information structure printed according to the invention in the form of a QR code can increase patient safety and can further beneficially ensure a connection to the electronic patient file.

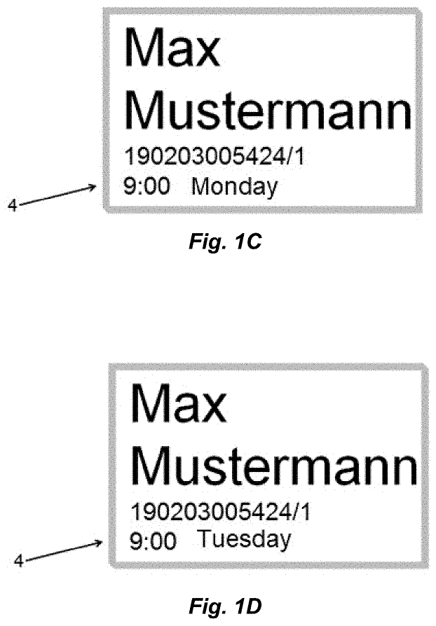

FIGS. 3A and 3B show schematic representations of top view of different exemplary dosage forms of the invention, wherein graphic printing patterns are applied, which serve particularly for children and geriatric patients in ensuring a simple identification of the drug and increasing compliance.

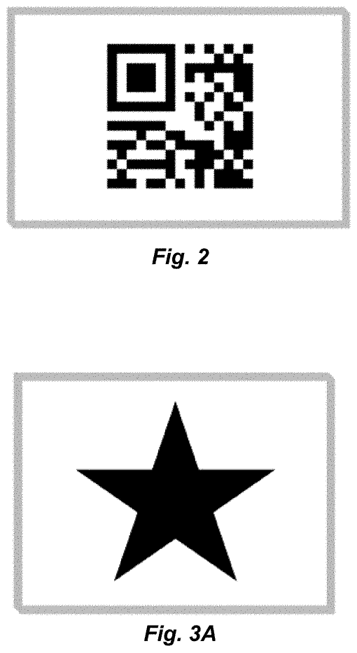

FIGS. 4A and 4B show schematic representations of top view of different exemplary dosage forms of the invention, wherein active agent-containing arrangements are applied on subareas. FIG. 4A shows a dosage form having printed areas of different dosages for subsequent adaptation. FIG. 4B shows a dosage form having printed areas with 2 different active agent concentrations or active agents.

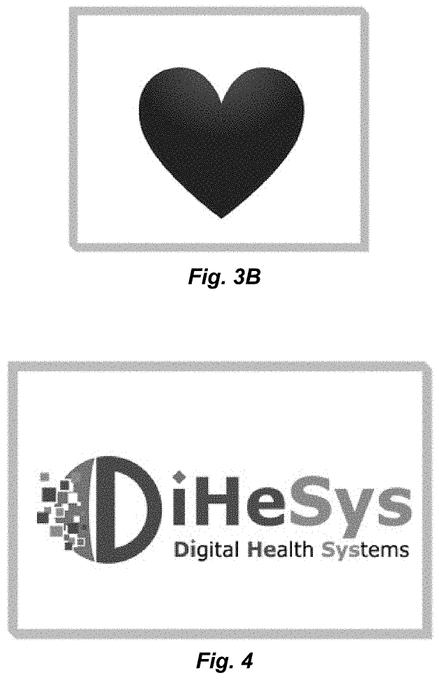

FIG. 5 shows a schematic representation of a top view of an exemplary dosage form of the invention, wherein an information structure is applied showing a company's logo. In a similar manner, signs of clinics, pharmaceutical companies, manufacturers or clinic logos can also be applied. Such dosage forms preferably serve to increase the identification of the patient with the unit using the respective sign.

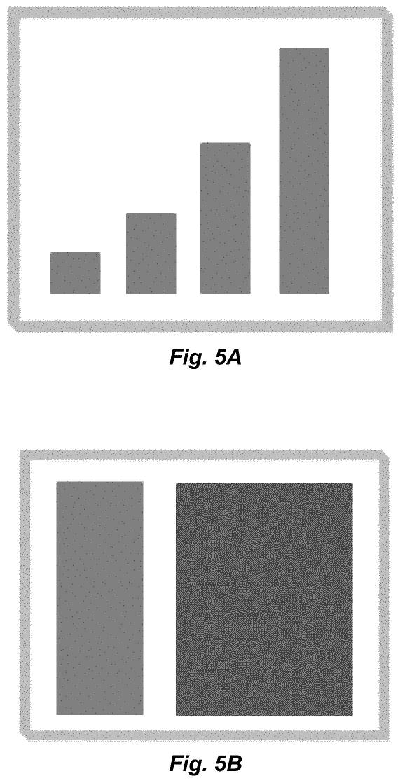

### DETAILED DESCRIPTION OF THE INVENTION

The present invention relates particularly to the following aspects and preferred embodiments:

Point 1. A method for producing a solid or semisolid pharmaceutical dosage form containing an active agent-free carrier structure and at least one pharmaceutical agent which is provided on at least a region of the carrier structure, the method comprising:
(i) providing at least one active agent-free carrier structure in a printing device designed for 2D and/or 3D printing of at least one pharmaceutical active agent; and
(ii) applying at least one pharmaceutical active agent by 2D and/or 3D printing on at least one region of the surface of the carrier structure in the printing device.

Point 2. The method of point 1 wherein in step (ii) more than one active agent is applied, wherein the active agents are applied together in one step or in several separate partial steps.

Point 3. The method of point 2 wherein the active agents are applied onto to the same or different regions of the carrier structure.

Point 4. The method of point 3 wherein the active agents are applied within a region separated from one another or spatially one above the other.

Point 5. The method according to any one of the preceding points wherein the method further comprises applying at least one colored substance by 2D and/or 3D printing onto at least one region of the carrier structure such that the applied substance forms at least one information structure visible on the carrier structure.

Point 6. The method of point 5 wherein the at least one colored substance is applied together with the at least one pharmaceutical active agent.

Point 7. The method of point 5 or 6 wherein the at least one information structure encodes information on the kind or the nature of the active agents applied onto the carrier structure and/or on the amount(s) of the active agent(s) applied onto the carrier structure and/or on the intended time point or intended time period of taking of the dosage form and/or the intended date of taking of the dosage form and/or on patient-related data and/or on the cost center and/or on the attending physician and/or on the pharmaceutical company providing the dosage form and/or on the medical unit dispensing the dosage form.

Point 8. The method of claim point 7 wherein the patient-related data are selected from the group consisting of name, age, sex, medication, and diseases of the patient.

Point 9. The method according to any one of points 5 to 8 wherein the information structure is selected from the group consisting of QR codes, characters, and numbers.

Point 10. The method according to any one of preceding points wherein the carrier structure is present as a tablet, capsule, suppository, plaster or thin film.

Point 11. The method of point 10 wherein the tablet is selected from the group consisting of oblong shape tablets, lozenges, implantable tablets, multiapplication tablets, disperse tablets, retard tablets, vaginal tablets, eye tablets, coated tablets, matrix tablets, chewable tablets, film-coated tablets, modified-release tablets, lacquer tablets, and enteric coated tablets.

Point 12. The method according to any one of preceding points wherein the method is a filament fused filament fabrication (FFF) method or a fusion layer modeling (FLM) method.

Point 13. The method of point 12 wherein the active agent(s) and the optional colored substance(s) are present in one filament or different filaments which comprise(s) a filament carrier substance into which the active agent(s) and the optional colored substance(s) is/are embedded.

Point 14. The method according to any one of points 1 to 11 wherein the method is a binder jetting method.

Point 15. The method of point 14 wherein the active agent(s) and the optional colored substance(s) are present in one or more powder carrier substances.

Point 16. The method according to any one of points 1 to 11 wherein in step (ii) single volume increments of a fluid are applied, wherein at least part of the applied volume increments of the fluid contains the active agent(s) and the optional color substance(s), and wherein the volume increments solidify after application.

Point 17. The method of point 16 wherein the volume increments are applied layer wise such that they contact each other at least partially.

Point 18. The method of point 16 or 17 wherein the fluid is a molten material or a liquid.

Point 19. The method according to any one of points 1 to 11 wherein step (ii) comprises the following partial steps:
applying a solution, suspension or emulsion containing the agent or agents onto at least a region of the carrier substance; and
evaporating the solution, suspension or emulsion such that the active agent(s) remain on the at least one region of the carrier structure.

Point 20. The method of point 19 wherein the carrier structure absorbs part of the solution, suspension or emulsion during the evaporation step.

Point 21. A solid or semisolid pharmaceutical dosage form which contains at least one pharmaceutical active agent and is prepared by the method according to any one of the points.

Point 22. A solid or semisolid dosage form comprising an active agent-free carrier structure onto which at least a two- and/or three-dimensional arrangement containing at least one pharmaceutical active agent is applied at least region wise.

Point 23. The dosage form of point 22 wherein more than one active agent is applied onto the carrier structure.

Point 24. The dosage form of point 23 wherein the active agents are applied on the same or different regions on the carrier structure.

Point 25. The dosage form of point 24 wherein the active agents are applied with a region separated from one another or spatially one above the other.

Point 26. The dosage form according to any one of points 23 to 25 wherein at least one colored substance is further applied onto at least one region of the carrier structure such that the applied substance forms at least one information structure visible on the carrier structure.

Point 27. The dosage form of point 26 wherein the at least on information structure encodes information on the kind or the nature of the active agents applied onto the carrier structure and/or on the amount(s) of the active agent(s) applied onto the carrier structure and/or on the intended time point or intended time period of taking of the dosage form and/or the intended date of taking of the dosage form and/or on patient-related data and/or on the cost center and/or on the attending physician and/or on the pharmaceutical company providing the dosage form and/or on the medical unit dispensing the dosage form.

Point 28. The dosage form of point 27 wherein patient-related data is selected form the group consisting of name, age, sex, medication, and diseases of the patient.

Point 29. The dosage form according to any one of claims 26 to 28 wherein the information structure is selected from the group consisting of QR codes, characters, and numbers.

Point 30. The dosage form according to any one of claims 23 to 29 being present as a tablet, capsule, suppository, plaster or thin film.

Point 31. The dosage form of claim 30 wherein the tablet is selected from the group consisting of oblong shape tablets, lozenges, implantable tablets, multiapplication tablets, disperse tablets, retard tablets, vaginal tablets, eye tablets, coated tablets, matrix tablets, chewable tablets, film-coated tablets, modified-release tablets, lacquer tablets, and enteric coated tablets.

1. A method for producing a solid or semisolid pharmaceutical dosage form containing an active agent-free carrier structure and at least one pharmaceutical agent which is provided on at least a region of the carrier structure, the method comprising:
(i) providing at least one active agent-free carrier structure in a printing device designed for 2D and/or 3D printing of at least one pharmaceutical active agent; and
(ii) applying at least one pharmaceutical active agent by 2D and/or 3D printing on at least one region of the surface of the carrier structure in the printing device.

2. The method of claim 1 wherein in step (ii) more than one active agent is applied, wherein the active agents are applied together in one step or in several separate partial steps.

3. The method of claim 2 wherein the active agents are applied onto the same at least one region or different regions of the carrier structure.

4. The method of claim 3 wherein the active agents are applied within a region separated from one another or spatially one above the other.

5. The method according to claim 1 wherein the method further comprises applying at least one colored substance by 2D and/or 3D printing onto at least one region of the carrier structure such that the applied substance forms at least one information structure visible on the carrier structure.

6. The method of claim 5 wherein the at least one colored substance is applied together with the at least one pharmaceutical active agent.

7. The method of claim 5 wherein the at least one information structure encodes information on the kind or the nature of the active agents applied onto the carrier structure and/or on the amount(s) of the active agent(s) applied onto the carrier structure and/or on the intended time point or intended time period of taking of the dosage form and/or the intended date of taking of the dosage form and/or on patient-related data and/or on the cost center and/or on the attending physician and/or on the pharmaceutical company providing the dosage form and/or on the medical unit dispensing the dosage form.

8. The method of claim 7 wherein the patient-related data are selected from the group consisting of name, age, sex, medication, and diseases of the patient.

9. The method claim 5 wherein the information structure is selected from the group consisting of QR codes, characters, and numbers.

10. The method according to claim 1 wherein the carrier structure is present as a tablet, capsule, suppository, plaster or thin film.

11. The method of claim 10 wherein the tablet is selected from the group consisting of oblong shape tablets, lozenges, implantable tablets, multiapplication tablets, disperse tablets, retard tablets, vaginal tablets, eye tablets, coated tablets, matrix tablets, chewable tablets, film-coated tablets, modified-release tablets, lacquer tablets, and enteric coated tablets.

12. The method according to claim 1 wherein the method is a filament fused filament fabrication (FFF) method or a fusion layer modeling (FLM) method.

13. The method of claim 12 where the active agent(s) and the optional colored substance(s) are present in one filament or different filaments which comprise(s) a filament carrier substance into which the active agent(s) and the optional colored substance(s) is/are embedded.

14. The method according to claim 1 wherein the method is a binder jetting method.

15. The method of claim 14 wherein the active agent(s) and the optional colored substance(s) are present in one or more powder carrier substances.

16. The method according to claim 1 wherein in step (ii) single volume increments of a fluid are applied, wherein at least part of the applied volume increments of the fluid contains the active agent(s) and the optional color substance(s), and wherein the volume increments solidify after application.

17. The method of claim 16 wherein the volume increments are applied layer wise such that they contact each other at least partially.

18. The method of claim 16 wherein the fluid is a molten material or a liquid.

19. The method according to claim 1 wherein step (ii) comprises the following partial steps:
applying a solution, suspension or emulsion containing the agent or agents onto at least a region of the carrier substance; and
evaporating the solution, suspension or emulsion such that the active agent(s) remain on the at least one region of the carrier structure.

20. The method of claim 19 wherein the carrier structure absorbs part of the solution, suspension or emulsion during the evaporation step.

21. A solid or semisolid pharmaceutical dosage form which contains at least one pharmaceutical active agent and is prepared by the method according to claim 1.

22. A solid or semisolid dosage form comprising an active agent-free carrier structure onto which at least a two- and/or three-dimensional arrangement containing at least one pharmaceutical active agent is applied at least region wise.

23. The dosage form of claim 22 wherein more than one active agent is applied onto the carrier structure.

24. The dosage form of claim 23 wherein the active agents are applied on the same or different regions on the carrier structure.

25. The dosage form of claim 24 wherein the active agents are applied with a region separated from one another or spatially one above the other.

26. The dosage form according to claim 23 wherein at least one colored substance is further applied onto at least one region of the carrier structure such that the applied substance forms at least one information structure visible on the carrier structure.

27. The dosage form of claim 26 wherein the at least on information structure encodes information on the kind or the nature of the active agents applied onto the carrier structure and/or on the amount(s) of the active agent(s) applied onto the carrier structure and/or on the intended time point or intended time period of taking of the dosage form and/or the intended date of taking of the dosage form and/or on patient-related data and/or on the cost center and/or on the attending physician and/or on the pharmaceutical company providing the dosage form and/or on the medical unit dispensing the dosage form.

28. The dosage form of claim 27 wherein patient-related data is selected form the group consisting of name, age, sex, medication, and diseases of the patient.

29. The dosage form according to claim 26 wherein the information structure is selected from the group consisting of QR codes, characters, and numbers.

30. The dosage form according to claim 23 being present as a tablet, capsule, suppository, plaster or thin film.

31. The dosage form of claim 30 wherein the tablet is selected from the group consisting of oblong shape tablets, lozenges, implantable tablets, multiapplication tablets, disperse tablets, retard tablets, vaginal tablets, eye tablets, coated tablets, matrix tablets, chewable tablets, film-coated tablets, modified-release tablets, lacquer tablets, and enteric coated tablets.
